(19) United States
(12) Patent Application Publication
(10) Pub. No.: US 2025/0104810 A1
(43) Pub. Date: Mar. 27, 2025

(54) GENE MINING METHOD AND SYSTEM BASED ON TRANSCRIPTOME AND DNA METHYLOME

(71) Applicant: Yangzhou University, Yangzhou (CN)

(72) Inventors: Xiaodong Yang, Yangzhou (CN); Lili Zhang, Yangzhou (CN); Lei Qiu, Yangzhou (CN); Jieni Gu, Yangzhou (CN); Ziyi Li, Yangzhou (CN); Xuehao Chen, Yangzhou (CN)

(21) Appl. No.: 18/914,164

(22) Filed: Oct. 12, 2024

(30) Foreign Application Priority Data
Nov. 16, 2023 (CN) 202311536372.1

(51) Int. Cl. G16B 25/10; C12Q 1/6869; G16B 30/00; G16B 40/20

(52) U.S. Cl. CPC G16B 25/10; C12Q 1/6869; G16B 30/00; G16B 40/20

(57) ABSTRACT

The present invention discloses a gene mining method and system based on transcriptome and DNA methylome. The gene mining method comprises: acquiring samples; performing transcriptome sequencing on the samples and analyzing data obtained by the sequencing to obtain differentially expressed genes; performing DNA methylation sequencing on the samples and analyzing data obtained by the sequencing to obtain differentially methylated genes; performing gene function enrichment on the obtained differentially expressed genes and differentially methylated genes to obtain a molecular protein-protein interaction (PPI) network; and performing gene clustering with the molecular PPI network to identify target genes. The present invention solves the problems that it takes time and effort to mine core genes of existing transcriptome and methylome and the result is not accurate enough. The present invention mines target genes by organically integrating originally isolated transcriptome data and DNA methylome data.

### TECHNICAL FIELD

The present invention relates a gene mining method and system based on transcriptome and DNA methylome and belongs to the technical field of transcriptomics and DNA methylomics.

### BACKGROUND

DNA methylation, an important epigenetic modification, has been extensively studied for the effect thereof on gene transcription regulation in eukaryotes. With the continuous improvement of second generation sequencing technology and the reduction of cost, whole genome bisulfite sequencing (WGBS), as a high resolution DNA methylation sequencing technology, has been widely used to study the effect of DNA methylation on gene regulation. As the amount of available WGBS data continues to increase, the analysis of genome-wide methylome datasets has become a bottleneck in resolving the influence of DNA methylation variation on gene expression.

The current analysis methods for DNA methylome data have encountered at least three major challenges:
First, DNA methylation is dynamic and can respond to environmental changes. Therefore, in biological experiments, it is necessary to distinguish the background noise of the system from the biologically significant changes in response to environmental signals. The process of differentiation is very challenging for conventional analysis methods.
Second, due to differences in methods and parameter setting of methylome analysis programs, experimental data is influenced by filtering methods or subjective user setting. Parameter setting that is too strict leads to information loss and excessive false negatives, while setting that is too loose leads to unacceptable false positive rates, thus affecting the downstream biological function analysis.
Third, the DNA methylation levels of individuals in biological samples vary greatly during development and environmental response, and traditional technical means lacks a reasonable treatment method for DNA methylation differences of the individuals.

Similarly, the analysis and verification of results of transcriptome also have the following two problems:
First, more differentially expressed genes are identified due to data pre-processing problems of selecting an inappropriate normalization method, setting unreasonable filter conditions and not considering batch effects as well as biological noise effects, and further screening of the differentially expressed genes is needed.
Second, gene expression is a dynamic and random process, resulting in heterogeneity in the expression of the same gene in a population. The existing researches indicate that for many genes, RNA molecules are not produced at a constant rate even if the regulation signals remain constant.

Meanwhile, a forward genetics approaches was often used to mine genes related to specific traits. Traditional forward genetics approaches usually have some problems and limitations:
The first is large consumption of time and resources. The traditional forward genetics approaches consume a lot of time and resources. It usually takes many years to separate and screen from varietal hybridization and finally identify genes related to the target traits. Such cumbersome process makes a breeding cycle very long.
The second is limited genetic diversity. The traditional forward genetics approaches often rely on natural or artificially hybridized materials, which may limit the available genetic diversity. Sometimes, breeders may not be able to obtain appropriate parents with desired characteristics, thus limiting the feasibility of forward genetics mapping.
The third is gene interaction and environment interaction. Plant traits are influenced by multiple genes which may interact with each other. In addition, environmental conditions can also affect the performance of traits. The traditional methods are difficult to consider both gene interaction and environment interaction, so it is difficult to accurately mine genes related to traits.

The Chinese invention patent CN201910608866.3 records a transcriptome and DNA methylome data association analysis method and system, and the method solves the problems of one-sidedness of single omics sequencing data analysis and unreliability of partial data in the prior art. However, the method has the problems of low classification accuracy, insensitivity to outliers, weak robustness and fault tolerance to noisy data and low data analysis efficiency.

### SUMMARY

The purpose of the present invention is to overcome the defects of the prior art and provide a gene mining method and system based on transcriptome and DNA methylome, which can mine target genes by integrating transcriptome data and DNA methylome data.

To achieve the above purpose, the present invention adopts the following technical solution:

The present invention provides a gene mining method based on transcriptome and DNA methylome, comprising the following steps:
Acquiring samples;
Performing transcriptome sequencing on the samples and analyzing data obtained by the sequencing to obtain differentially expressed genes;
Performing DNA methylation sequencing on the samples and analyzing data obtained by the sequencing to obtain differentially methylated genes;
Performing gene function enrichment on the obtained differentially expressed genes and differentially methylated genes to obtain a molecular protein-protein interaction (PPI) network;
Performing gene clustering with the molecular PPI network to identify target genes.

Further, tools for the gene function enrichment comprise DAVID, Enrichr, Metascape and GSEA;
Reference databases for the gene function enrichment comprise GO, KEGG and Reactome.

Further, the molecular PPI network is a molecular PPI network of genes contained in an enrichment pathway shared by the differentially expressed genes and the differentially methylated genes.

Further, the step of performing gene clustering with the molecular PPI network to identify target genes comprises the following steps:
Calculating the betweenness centrality, closeness centrality, average shortest path length and clustering coefficient of protein encoded by the differentially methylated genes and the differentially expressed genes in the molecular PPI network to obtain positions and relationships of the differentially methylated genes and the differentially expressed genes in molecular PPI network structure;
Based on a K-means clustering method, carrying out feature selection according to the differences of the betweenness centrality, closeness centrality, average shortest path length and clustering coefficient, and dividing the differentially methylated genes and the differentially expressed genes into clusters with similar network features;
Taking a cluster with the maximum sum of the betweenness centrality of nodes as a target cluster, and taking genes in the target cluster as the target genes.

Further, the betweenness centrality comprises the following formula:

$${C}_{B} ( υ ) = {\sum }_{δ \neq υ \neq t} \frac{d ( υ , u )}{{σ}_{ST}} ;$$

wherein C_{B}(υ) is the betweenness centrality of a node υ in the molecular PPI network, σ_{ST} is the number of shortest paths from a node δ to a node t in the molecular PPI network, d(υ,u) is the number of shortest paths from the node υ to other nodes in the molecular PPI network, and u is traversal from the node υ to other nodes in the molecular PPI network.

Further, the closeness centrality comprises the following formula:

$${C}_{C} ( υ ) = \frac{1}{{\sum }_{u} d ( υ , u )} ;$$

wherein C_{C}(υ) is the closeness centrality of the node υ in the molecular PPI network, d(υ,u) is the number of shortest paths from the node υ to other nodes in the molecular PPI network, and u is traversal from the node υ to other nodes in the molecular PPI network.

Further, the average shortest path length comprises the following formula:

$$L = \frac{1}{n ( n - 1 )} {\sum }_{i \neq j} {d}_{i j} ;$$

wherein L is average shortest path length, n is the total number of nodes in the molecular PPI network, and dᵢⱼ is shortest path length from a node i to a node j in the molecular PPI network.

Further, the clustering coefficient comprises the following formula:

$$C ( υ ) = \frac{2 \times number of triangles centered on υ}{degree of υ \times ( degree of υ - 1 )} ;$$

wherein C(υ) is the clustering coefficient of the node υ in the molecular PPI network, number of triangles centered on υ is the number of triangles centered on the node υ, and degree of υ is the degree of the node υ in the molecular PPI network.

Further, the step of performing DNA methylation sequencing on the samples and analyzing data obtained by the sequencing to obtain differentially methylated genes comprises:
Comparing DNA methylation data with reference genome to obtain basic methylated signals;
Based on a Bayes machine learning method, calculating Hellinger divergence by using the DNA methylation level of the samples and the DNA methylation level of control samples with reference to the DNA methylation level of control samples, and fitting a statistical model by the Hellinger divergence according to the Akaike information criterion and the cross-validation correlation coefficient of nonlinear regression;
Distinguishing methylated signals and background variation signals by the statistical model to screen out cytosine positions with methylated signals, and determining differentially methylated positions according to the cytosine positions with methylated signals and thresholds of the differentially methylated positions.

On the other hand, the present invention provides a gene mining system based on transcriptome and DNA methylome, comprising:
A sample acquisition module used for acquiring samples;
A differentially expressed gene acquisition module used for performing transcriptome sequencing on the samples and analyzing data obtained by the sequencing to obtain differentially expressed genes;
A differentially methylated gene acquisition module used for performing DNA methylation sequencing on the samples and analyzing data obtained by the sequencing to obtain differentially methylated genes;
A molecular PPI network construction module used for performing gene function enrichment on the obtained differentially expressed genes and differentially methylated genes to obtain a molecular PPI network;
A target gene identification module used for performing gene clustering with the molecular PPI network to identify target genes.

Compared with the prior art, the present invention has the following beneficial effects:

The present invention distinguishes background signals of natural variation and induced variation based on the distribution of DNA methylome data to accurately identify background noise and effectively remove the background noise, eliminating thermodynamic fluctuations without biological significance.

Based on the application of the Yoden index, the present invention constructs optimal thresholds of differentially methylated positions, which can avoid loss of effective signals and generation of false positives.

The present invention can accurately and efficiently screen out target genes by constructing the molecular PPI network for gene clustering identification.

The present invention jointly analyzes methylome data and transcriptome data to provide a more comprehensive interpretation of data results and exclude the heterogeneity of isogenic populations caused by transcriptional burst, and the analysis results are flexible and reliable and are closer to the real biological regulation mechanism.

The present invention can mine target genes by integrating transcriptome data and DNA methylome data to realize joint analysis of methyltome data and transcriptome data, have a more comprehensive interpretation of data results, and exclude the heterogeneity of isogenic populations caused by transcriptional burst, which can not only greatly improve the mining efficiency, but also effectively reduce the proportion of false positives, avoid signal loss and improve the accuracy of results.

### DESCRIPTION OF DRAWINGS

FIG. 1 is a flow chart of one embodiment of a gene mining method based on transcriptome and DNA methylome of the present invention;

FIG. 2 is a flow chart of one embodiment of a gene mining method based on transcriptome and DNA methylome of the present invention;

FIG. 3 shows a molecular PPI network of differentially expressed genes and differentially methylated genes in cucumber morphogenesis-related tissues in embodiment 4;

FIG. 4 (a) shows single base cytosine DNA methylation of CsLSH6 in seven tissues of cucumber in embodiment 4;

FIG. 4 (b) shows expression levels of CsLSH6 in seven tissues of cucumber in embodiment 4;

FIG. 4 (c) shows single base cytosine DNA methylation of CsIAA16 in seven tissues of cucumber in embodiment 4;

FIG. 4 (d) shows expression levels of CsIAA16 in seven tissues of cucumber in embodiment 4;

FIG. 5 shows a molecular PPI network of differentially expressed genes and differentially methylated genes in a process of female and male flower differentiation of cucumber in embodiment 5;

FIG. 6 (a) shows DNA methylation of CsAP1 at a single base cytosine position in male and female flowers in embodiment 5;

FIG. 6 (b) shows expression levels of CsAP1 in seven different tissues in embodiment 5;

FIG. 6 (c) shows DNA methylation of CsDTX55 at a single base cytosine position in male and female flowers in embodiment 5;

FIG. 6 (d) shows expression levels of CsDTX55 in seven different tissues in embodiment 5;

FIG. 7 is a schematic diagram of a signal detection theory of methylation analysis of the present invention.

### DETAILED DESCRIPTION

The present invention will be further described below in combination with the drawings. The following embodiments are only used for illustrating the technical solution of the present invention more clearly, not used for limiting the protection scope of the present invention.

### Embodiment 1

The present embodiment introduces a gene mining method based on transcriptome and DNA methylome.

The gene mining method based on transcriptome and DNA methylome of the present embodiment comprises the following steps:
S1: Acquiring samples;
S2: Performing transcriptome sequencing on the samples and analyzing data obtained by the sequencing to obtain differentially expressed genes;
S3: Performing DNA methylation sequencing on the samples and analyzing data obtained by the sequencing to obtain differentially methylated genes;
S4: Performing gene function enrichment on the obtained differentially expressed genes and differentially methylated genes to obtain a molecular PPI network;
S5: Performing gene clustering with the molecular PPI network to identify target genes.

### Embodiment 2

On the basis of embodiment 1, the present embodiment introduces a gene mining method based on transcriptome and DNA methylome in detail.

The gene mining method based on transcriptome and DNA methylome of the present embodiment comprises the following steps:
S1: Acquiring samples.
S2: Performing transcriptome sequencing on the samples and analyzing data obtained by the sequencing to obtain differentially expressed genes.

In application, step S2 comprises the following steps:
Comparing DNA methylation data with reference genome to obtain basic methylated signals;
Based on a Bayes machine learning method, calculating Hellinger divergence by using the DNA methylation level of the samples and the DNA methylation level of control samples with reference to the DNA methylation level of control samples, and fitting a statistical model by the Hellinger divergence according to the Akaike information criterion and the cross-validation correlation coefficient of nonlinear regression;
Distinguishing methylated signals and background variation signals by the statistical model to screen out cytosine positions with methylated signals, and determining differentially methylated positions according to the cytosine positions with methylated signals and thresholds of the differentially methylated positions.
S3: Performing DNA methylation sequencing on the samples and analyzing data obtained by the sequencing to obtain differentially methylated genes.
S4: Performing gene function enrichment on the obtained differentially expressed genes and differentially methylated genes to obtain a molecular PPI network.

In application, tools for the gene function enrichment comprise DAVID, Enrichr, Metascape and GSEA; and reference databases for the gene function enrichment comprise GO, KEGG and Reactome.
S5: Performing gene clustering with the molecular PPI network to identify target genes.

In application, the molecular PPI network is a molecular PPI network of genes contained in an enrichment pathway shared by the differentially expressed genes and the differentially methylated genes.

In practical application, step S5 comprises the following steps:
Calculating the betweenness centrality, closeness centrality, average shortest path length and clustering coefficient of protein encoded by the differentially methylated genes and the differentially expressed genes in the molecular PPI network to obtain positions and relationships of the differentially methylated genes and the differentially expressed genes in molecular PPI network structure;
Based on a K-means clustering method, carrying out feature selection according to the differences of the betweenness centrality, closeness centrality, average shortest path length and clustering coefficient, and dividing the differentially methylated genes and the differentially expressed genes into clusters with similar network features;
Taking a cluster with the maximum sum of the betweenness centrality of nodes as a target cluster, and taking genes in the target cluster as the target genes.

The betweenness centrality of the present embodiment comprises the following formula:

$${C}_{B} ( υ ) = {\sum }_{δ \neq υ \neq t} \frac{d ( υ , u )}{{σ}_{ST}} ;$$

wherein C_{B}(υ) is the betweenness centrality of a node υ in the molecular PPI network, σ_{ST} is the number of shortest paths from a node δ to a node t in the molecular PPI network, d(υ,u) is the number of shortest paths from the node υ to other nodes in the molecular PPI network, and u is traversal from the node υ to other nodes in the molecular PPI network.

The closeness centrality of the present embodiment comprises the following formula:

$${C}_{C} ( υ ) = \frac{1}{{\sum }_{u} d ( υ , u )} ;$$

wherein C_{C}(υ) is the closeness centrality of the node υ in the molecular PPI network, d(υ,u) is the number of shortest paths from the node υ to other nodes in the molecular PPI network, and u is traversal from the node υ to other nodes in the molecular PPI network.

The average shortest path length of the present embodiment comprises the following formula:

$$L = \frac{1}{n ( n - 1 )} {\sum }_{i \neq j} {d}_{i j} ;$$

wherein L is average shortest path length, n is the total number of nodes in the molecular PPI network, and dᵢⱼ is shortest path length from a node i to a node j in the molecular PPI network.

The clustering coefficient of the present embodiment comprises the following formula:

$$C ( υ ) = \frac{2 \times number of triangles centered on υ}{degree of υ \times ( degree of υ - 1 )} ;$$

wherein C(υ) is the clustering coefficient of the node υ in the molecular PPI network, number of triangles centered on υ is the number of triangles centered on the node υ, and degree of u is the degree of the node υ in the molecular PPI network.

### Embodiment 3

On the basis of embodiment 1 or 2, the present embodiment introduces a gene mining method based on transcriptome and DNA methylome in detail.

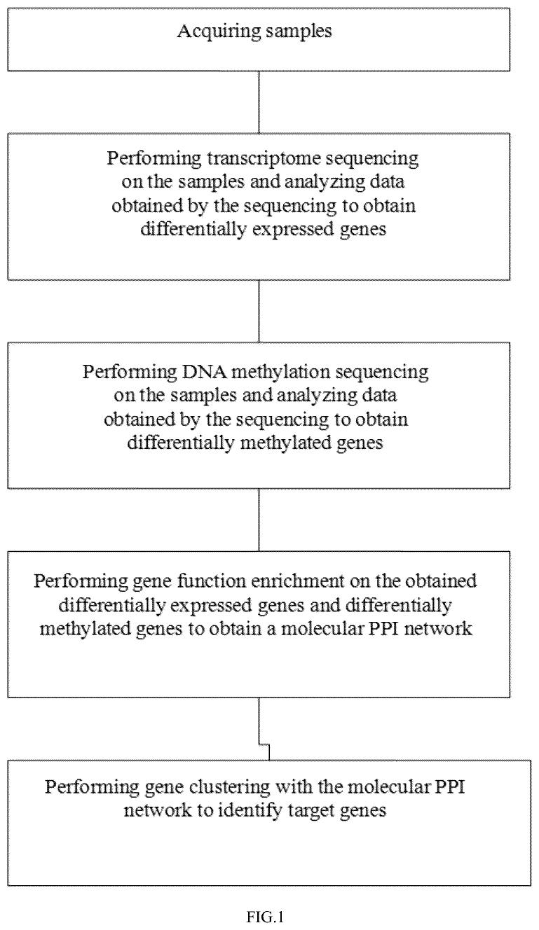

The gene mining method based on transcriptome and DNA methylome of the present embodiment comprises the following steps, as shown in FIG. 1:
S1: Acquiring samples.
S2: Performing transcriptome sequencing on the samples and analyzing data obtained by the sequencing to obtain differentially expressed genes.
S3: Performing DNA methylation sequencing on the samples and analyzing data obtained by the sequencing to obtain differentially methylated genes.

In application, DNA methylation sequencing is whole genome DNA methylation sequencing.

In practical application, step S3 comprises the following content:
S31: Performing quality control on methylation data, including checking data quality and removing data with sequencing coverage less than 4 times, and comparing the methylation data with reference genome to obtain basic methylated signals.
S32: Calculating Hellinger divergence by using the DNA methylation levels of control and treated samples with reference to the DNA methylation levels of the control samples, and making correction using a Bayes algorithm.

In application, the selection of the control samples can be adjusted according to the experimental design, and most of the control samples are wild-type samples in the control samples and materials in the experimental design.

In practical application, the comparison of data for consecutive time points can also be the sum or average of all the DNA methylation levels. The DNA methylation levels are the proportion of the number of reads obtained by the sequencing to the total number of reads.

The Hellinger divergence comprises the following formula:

$${H}^{D} ( p , q ) = {( \sqrt{p} , \sqrt{q} )}^{2} + {( \sqrt{1 - p} - \sqrt{1 - q} )}^{2} ;$$

wherein H^{D} is the Hellinger divergence, p is a methylation level of the control samples, and q is a methylation level of the samples.

The present embodiment uses a statistical model to distinguish signals from noise, quantifies the difference between probability distributions, and matches an optimal fitting model following two fitting metrics, namely the Akaike Information Criterion (AIC) and the cross-validation correlation coefficient of nonlinear regression (R.Cross.val) so as to construct a statistical model.

The fitting process of the statistical model comprises the following formula:

$${H}_{obs ( P )}^{D} = {\hat{β}}_{0} {H}_{obs ( P - 1 )}^{D} + {\hat{β}}_{1} \cdot {X}_{1 P} + {\hat{β}}_{0} \cdot {\hat{β}}_{1} \cdot {X}_{1 ( P - 1 )} + e ;$$

wherein H^{D} is the Hellinger divergence, P is a gene position, x is frequency, e is the residual of the model, and \hat{β}₀, \hat{β}₁ is the fitting coefficient vector of the statistical model.
S33: After obtaining a model with the highest degree of fitting, using biologically significant signals from the control group and the treatment group to determine optimal determination thresholds of the obtained differentially methylated positions, wherein the thresholds are used for distinguishing methylated signals from background variation.

In application, a method for determining the optimal thresholds of the differentially methylated positions is based on the application of the Yoden index. The Yoden index is defined as a statistical parameter commonly used to assess the overall performance of a binary classification test or model in medical diagnosis and classification tasks.

Above the thresholds are differentially methylated positions with biological significance, while below the thresholds are thermodynamic fluctuations without biological significance.

For the identification of the differentially methylated genes, positions with at least five DMPs and the DMP density of 0.0001 are selected. Subsequently, comparison among groups is performed using a likelihood ratio test (LRT) to identify positions with log 2fold change >1 and the adjusted p value <0.05.

The Bayes machine learning method is used to calculate the Hellinger divergence, distinguish signals from noise and quantify the difference between probability distributions. For the methylome analysis of morphogenesis-related tissues, the mean methylation counts of tendril, stem and petiole tissues are assigned as the treatment group, while the mean methylation counts of male flowers, female flowers, leaves and roots are used as controls.
3) Constructing a statistical model, following two fitting metrics, namely the Akaike Information Criterion (AIC) and the cross-validation correlation coefficient of nonlinear regression (R.Cross.val), to match an optimal fitting model so as to construct a statistical model, and using the statistical models to establish a relationship between methylated signals and background variation.
4) Screening out cytosine positions that may have methylated signals based on the results of the statistical model.
5) Using potential signals from the control group and the treatment group to determine optimal thresholds of the differentially methylated positions so as to screen out differentially methylated positions, wherein a method for determining the optimal thresholds of the differentially methylated positions is based on the application of the Yoden index.
6) Acquiring real methylated positions according to the optimal thresholds to screen differentially methylated genes.
S4: Performing gene function enrichment on the obtained differentially expressed genes and differentially methylated genes to obtain a molecular PPI network.

In application, tools for the gene function enrichment comprise Database for Annotation, Visualization and Integrated Discovery (DAVID), Enrichr, Metascape, gene set enrichment analysis (GSEA), etc.

Reference databases for the gene function enrichment comprise Gene Ontology (GO), Kyoto Encyclopedia of Genes and Genomes (KEGG), Reactome, etc.
S5: Performing gene clustering with the molecular PPI network to identify target genes.

In application, the molecular PPI network is a molecular PPI network of genes contained in an enrichment pathway shared by the differentially expressed genes and the differentially methylated genes.

In practical application, step S5 comprises the following steps:
S51: Calculating the betweenness centrality, closeness centrality, average shortest path length and clustering coefficient of protein encoded by the differentially methylated genes and the differentially expressed genes in the molecular PPI network to obtain positions and relationships of the differentially methylated genes and the differentially expressed genes in molecular PPI network structure;
S52: Based on a K-means clustering method, carrying out feature selection according to the differences of the betweenness centrality, closeness centrality, average shortest path length and clustering coefficient, and dividing the differentially methylated genes and the differentially expressed genes into clusters with similar network features;
S53: Taking a cluster with the maximum sum of the betweenness centrality of nodes as a target cluster, and taking genes in the target cluster as the target genes.

In practical application:

The betweenness centrality is defined as a frequency at which any node serves as a bridge or intermediary in the shortest path connecting other nodes in the molecular PPI network.

The closeness centrality is defined as average shortest path length from any node to all other nodes in the molecular PPI network.

The average shortest path length is defined as length of an average shortest path between any two nodes in the molecular PPI network.

The clustering coefficient is defined as a degree of interconnection between neighbor nodes of a node.

The degree is defined as the number of nodes directly connected to other nodes.

Eccentricity is defined as length of a shortest path from a node to other nodes in the molecular PPI network, and the maximum value of each shortest path is selected as the eccentricity of the node.

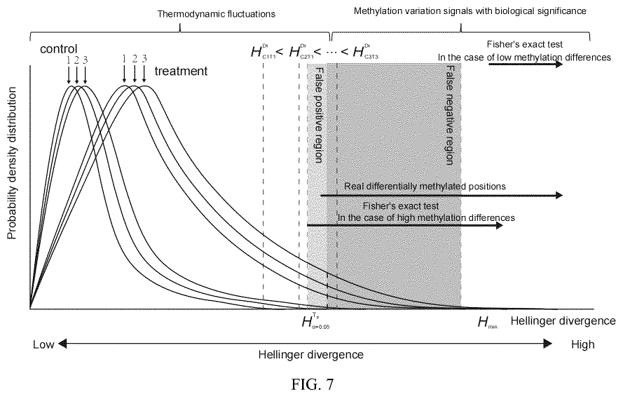

Referring to FIG. 7, the present embodiment identifies thresholds of the real differentially methylated positions (DMPs) based on the probability distribution of the methylated signals. The thresholds of the differentially methylated positions determined by a conventional Fisher's exact test tend to cause a large number of false positives in the case of high methylation differences and a large number of false negatives in the case of low methylation differences.

The present embodiment uses the Bayes machine learning method to calculate the Hellinger divergence, distinguishes thermodynamic fluctuations from methylation variation signals with biological significance, and quantifies the difference between probability distributions.

Based on the statistical model, the Yoden index is used to estimate the optimal thresholds of the differentially methylated positions to distinguish methylated signals caused by treatment from methylated signals of natural background variation, avoiding the large number of false positives and false negatives caused by the conventional Fisher's exact test.

### Embodiment 4

The present embodiment introduces a gene mining method for cucumber morphogenesis-related genes in detail.

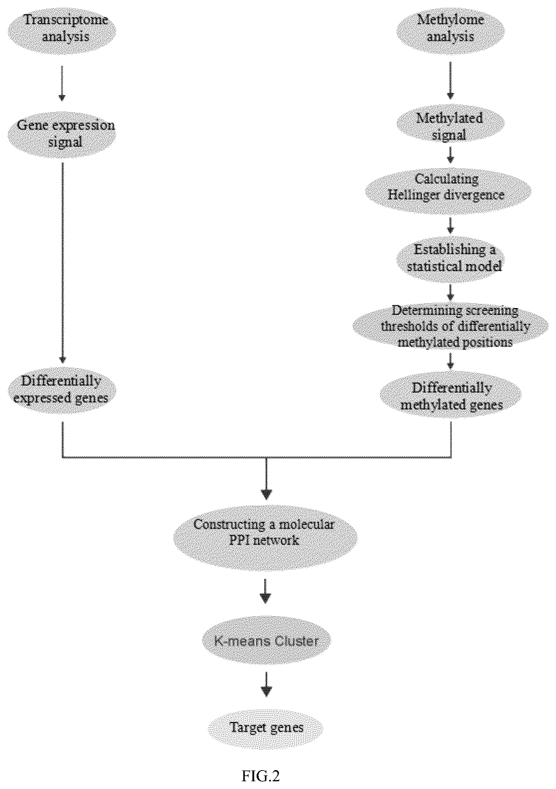

The gene mining method for cucumber morphogenesis-related genes of the present embodiment adopts the gene mining method recorded in any one of embodiments 1-3, as shown in FIG. 2.
(1) Acquiring samples.
(2) Performing transcriptome sequencing on the samples, and analyzing and processing data obtained by the sequencing to achieve collection and processing of differentially expressed gene data and methylation data.
(3) Performing DNA methylation sequencing on the samples, and analyzing and processing data obtained by the sequencing in combination with a machine learning algorithm to obtain differentially methylated genes.

In application, step (3) comprises the following steps:
1) Performing quality control on the methylation data, including checking data quality and removing low-quality sequencing data, and comparing the methylation data with reference genome to obtain basic methylated signals.
2) Calculating the Hellinger divergence by using the Bayes machine learning method with reference to the methylated signals of the control individuals, distinguishing signals from noise, and quantifying the difference between probability distributions. For the methylome analysis of morphogenesis-related tissues, the mean methylation counts of tendril, stem and petiole tissues are assigned as the treatment group, while the mean methylation counts of male flowers, female flowers, leaves and roots are used as controls.
3) Constructing a statistical model, and following two fitting metrics, namely the Akaike Information Criterion (AIC) and the cross-validation correlation coefficient of nonlinear regression (R.Cross.val), to match an optimal fitting model so as to construct a statistical model.
4) Screening out cytosine positions that may have methylated signals based on the results of the statistical model.
5) Using potential signals from the control group and the treatment group to determine optimal thresholds of the differentially methylated positions so as to screen out differentially methylated positions, wherein a method for determining the optimal thresholds of the differentially methylated positions is based on the application of the Yoden index.
6) Acquiring real methylated positions according to the optimal thresholds to screen differentially methylated genes.

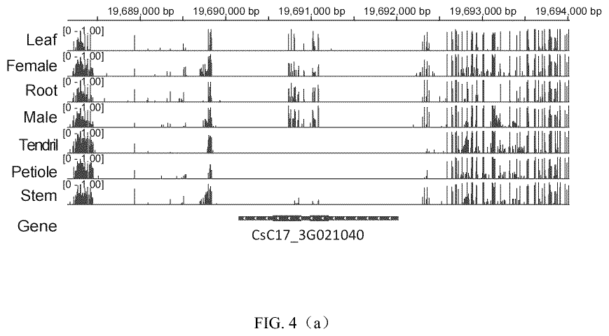

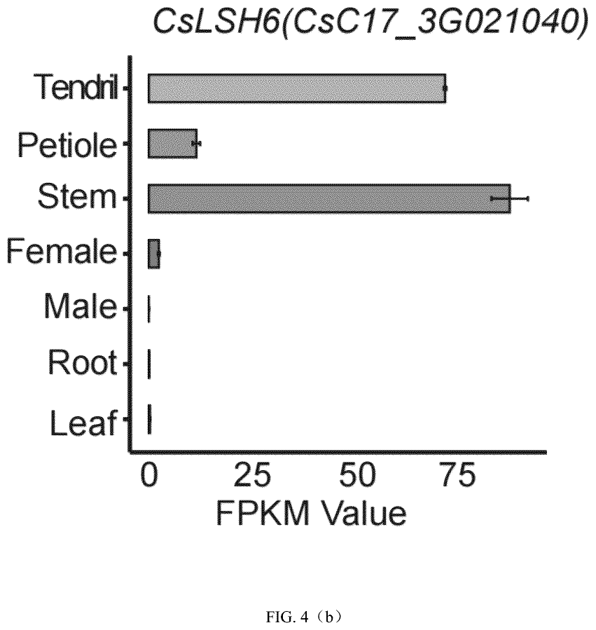

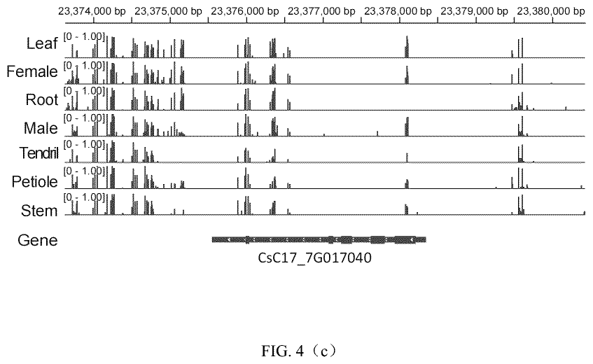

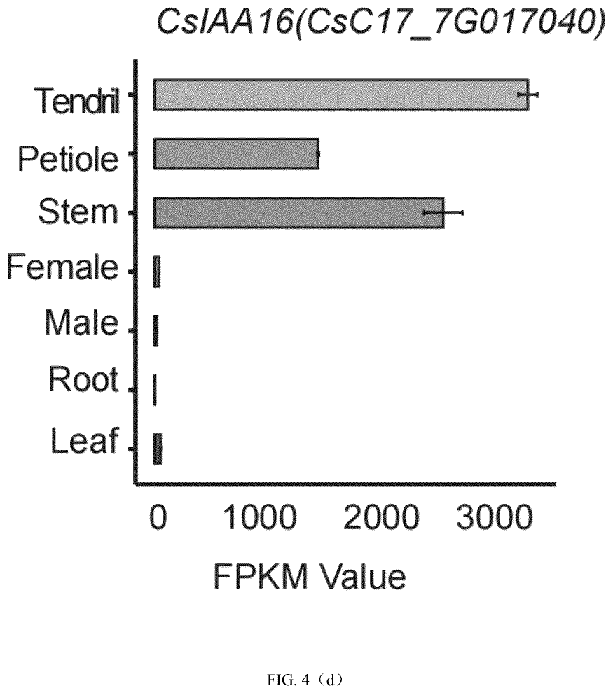

In practical application, the methylation level of each cytosine is calculated by using the average mC/(mC+uC) of each tissue, wherein mC represents methylated cytosine, and uC represents unmethylated cytosine. Patterns may vary slightly between different individuals due to methylation fluctuations, but only one representative plant is selected for each stage. The present embodiment detects single base cytosine DNA methylation of CsLSH6 and CsIAA16 in seven tissues, as shown in FIG. 4 (a) and FIG. 4 (c), and measures the expression levels of CsLSH6 and CsIAA16 in seven tissues, as shown in FIG. 4 (b) and FIG. 4 (d), which are described with an FPKM value.

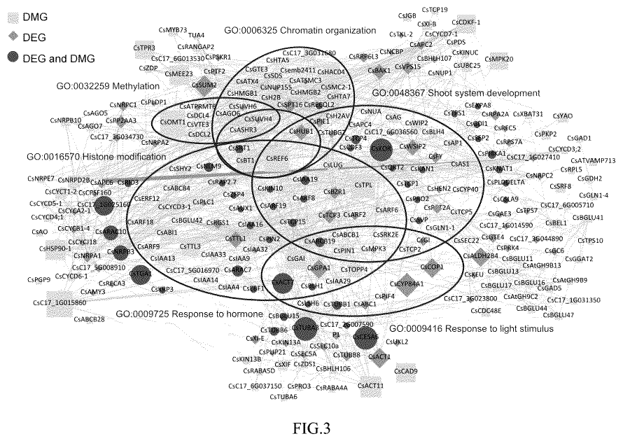

(4) Performing gene function enrichment on the obtained differentially expressed genes and differentially methylated genes, and selecting genes contained in an enrichment pathway shared by the differentially expressed genes and the differentially methylated genes to construct a molecular PPI network.
In application, the molecular PPI network is a molecular PPI network of differentially expressed genes and differentially methylated genes in cucumber morphogenesis-related tissues, as shown in FIG. 3.

2050 genes extracted from cucumber morphogenesis are derived from 73 enrichment pathways shared by differentially methylated genes and differentially expressed genes resulting from comparison of tendril, stem and petiole tissues with leaf, male flower, female flower and root tissues. K-means clustering based on machine learning is used to identify the 2050 genes, and the result is that 249 hub genes constituting subnetworks are identified. The size of each node in the network corresponds to the betweenness centrality thereof, which quantifies the influence on the information flow within the network. Different shapes represent different genotypes, differentially expressed genes are square, differentially methylated genes are diamond-shaped, and genes that are both differentially expressed genes and differentially methylated genes are circular.
(5) Performing gene clustering with the molecular PPI network to identify target genes.

### Embodiment 5

The present embodiment introduces a gene mining method in a process of female and male flower differentiation of cucumber in detail.

The gene mining method in a process of female and male flower differentiation of cucumber of the present embodiment adopts the gene mining method recorded in any one of embodiments 1-3.

In application, male flower tissues are set as samples, and female flower tissues are set as control samples.

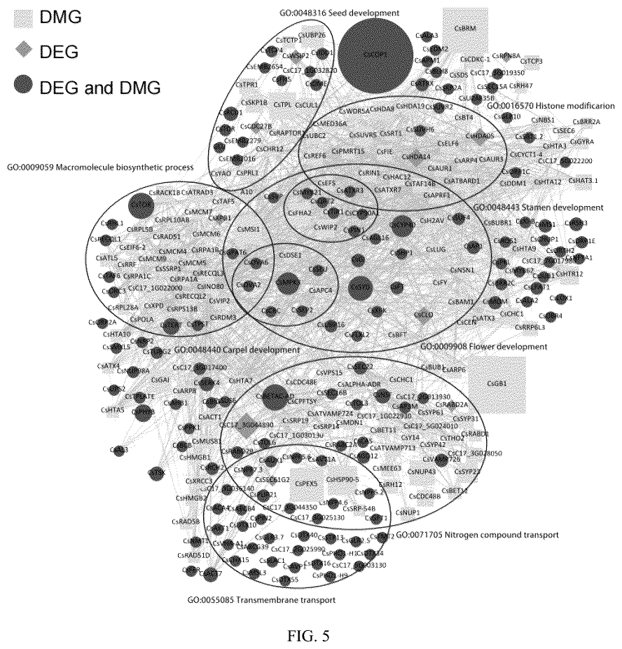

The molecular PPI network of the present embodiment is a molecular PPI network of differentially expressed genes and differentially methylated genes in the process of female and male flower differentiation of cucumber, as shown in FIG. 5. 3940 genes extracted from female and male flower differentiation are derived from 27 enrichment pathways shared by DMGs and DEGs resulting from comparison of female and male flowers. The K-means clustering method driven by machine learning is used to identify a cluster of closely related genes among the 3940 genes, totaling 297 core genes. Subsequently, for a deeper understanding of the functional correlation of the core gene subnetwork, gene enrichment analysis is performed. In the network, the size of each node corresponds to the betweenness centrality thereof, which is a measure of the influence of the information flow. Different shapes represent different genotypes, differentially expressed genes are square, differentially methylated genes are diamond-shaped, and genes that are both differentially expressed genes and differentially methylated genes are circular.

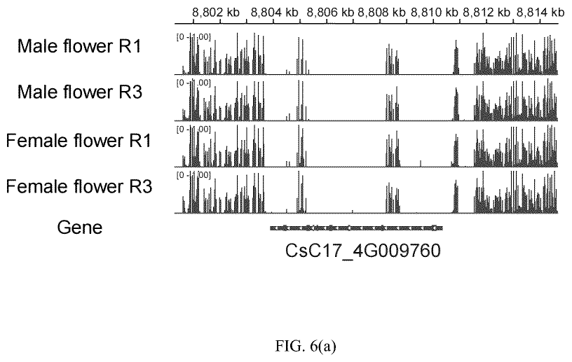

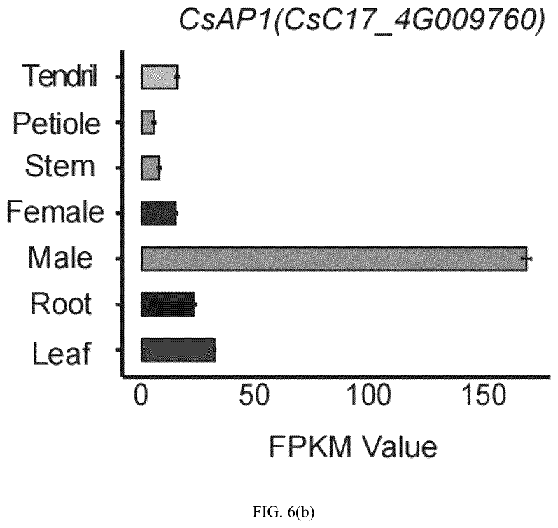

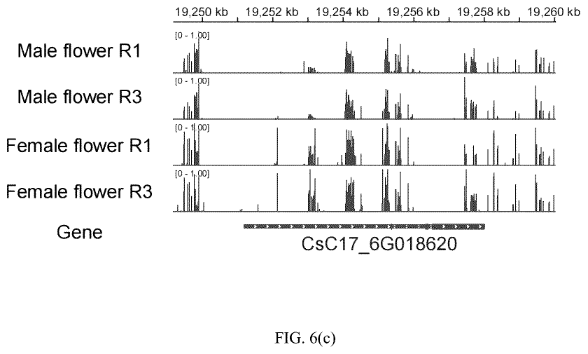

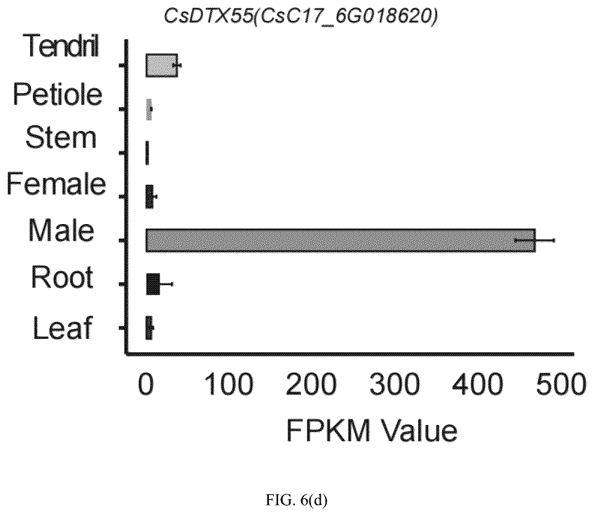

In practical application, the present embodiment detects DNA methylation of CsAP1 and CsDTX55 at single base cytosine positions in male and female flowers, as shown in FIG. 6 (a) and FIG. 6 (c). To determine changes in the methylation level at each cytosine position, the mean value of methylated cytosine (mC) for each tissue is divided by the sum of methylated and unmethylated cytosine (mC+uC). Such calculation involves comparison of male flowers with female flowers. The expression levels of CsAP1 and CsDTX55 in seven different tissues are quantified with the FPKM value, as shown in FIG. 6 (b) and FIG. 6 (d).

### Embodiment 6

The present embodiment introduces a gene mining system based on transcriptome and DNA methylome.

The gene mining system based on transcriptome and DNA methylome of the present embodiment comprises:
A sample acquisition module used for acquiring samples;
A differentially expressed gene acquisition module used for performing transcriptome sequencing on the samples and analyzing data obtained by the sequencing to obtain differentially expressed genes;
A differentially methylated gene acquisition module used for performing DNA methylation sequencing on the samples and analyzing data obtained by the sequencing to obtain differentially methylated genes;
A molecular PPI network construction module used for performing gene function enrichment on the obtained differentially expressed genes and differentially methylated genes to obtain a molecular PPI network;
A target gene identification module used for performing gene clustering with the molecular PPI network to identify target genes.

The specific functions of the above functional modules are realized according to the contents recorded in embodiments 1-4.

Those skilled in the art can apply the gene mining method of the present application in the following fields:
(1) Identifying association between methylated positions and gene expression.
(2) Finding methylated positions and regulatory genes related to important traits, designing molecular markers to select and improve disease resistance and stress resistance traits, and predicting disease resistance performance.
(3) Identifying methylated regulatory positions and genes related to stress tolerance as molecular markers.
(4) Speculating the influence of epigenetic modification on disease susceptibility genes, ascertaining the pathogenic mechanism, and providing new targets and therapeutic strategies.

Those skilled in the art should understand that the embodiments of the present application can provide a method, system or computer program product. Therefore, the present application can adopt a form of a full hardware embodiment, a full software embodiment or an embodiment combining software and hardware. Moreover, the present application can adopt a form of a computer program product capable of being implemented on one or more computer available storage media (including but not limited to disk memory, CD-ROM, optical memory, etc.) containing computer available program codes.

The present application is described with reference to flow charts and/or block diagrams according to the method, device (system) and computer program product in the embodiments of the present application. It should be understood that each flow and/or block in the flow charts and/or block diagrams and a combination of flows and/or blocks in the flow charts and/or block diagrams can be realized through computer program instructions. The computer program instructions can be provided for a processor of a general-purpose computer, a special-purpose computer, an embedded processor, or other programmable data processing devices to generate a machine, so that a device for realizing designated functions in one or more flows of the flow charts and/or one or more blocks of the block diagrams is generated through the instructions executed by the processor of the computer or other programmable data processing devices.

The computer program instructions can also be stored in a computer readable memory which can guide the computer or other programmable data processing devices to operate in a special mode, so that the instructions stored in the computer readable memory generate a manufactured product including an instruction device, the instruction device realizing designated functions in one or more flows of the flow charts and/or one or more blocks of the block diagrams.

The computer program instructions can also be loaded on the computer or other programmable data processing devices, so that a series of operation steps are executed on the computer or other programmable devices to generate processing realized by the computer. Therefore, the instructions executed on the computer or other programmable devices provide steps for realizing designated functions in one or more flows of the flow charts and/or one or more blocks of the block diagrams.

The embodiments of the present invention are described above with reference to the drawings, but the present invention is not limited to the above specific embodiments. The above specific embodiments are only illustrative, not restrictive. Under the enlightenment of the present invention, those ordinary skilled in the art can make many forms without departing from the purpose of the present invention and the protection scope of the claims, and these forms are protected by the present invention.

1. A gene mining method based on transcriptome and DNA methylome, comprising the following steps:
acquiring samples;
performing transcriptome sequencing on the samples and analyzing data obtained by the sequencing to obtain differentially expressed genes;
performing DNA methylation sequencing on the samples and analyzing data obtained by the sequencing to obtain differentially methylated genes, comprising:
comparing DNA methylation data with reference genome to obtain basic methylated signals;
based on a Bayes machine learning method, calculating Hellinger divergence by using the DNA methylation level of the samples and the DNA methylation level of control samples with reference to the DNA methylation level of control samples, and fitting a statistical model by the Hellinger divergence according to the Akaike information criterion and the cross-validation correlation coefficient of nonlinear regression;
distinguishing methylated signals and background variation signals by the statistical model to screen out cytosine positions with methylated signals, and determining differentially methylated positions according to the cytosine positions with methylated signals and thresholds of the differentially methylated positions;
performing gene function enrichment on the obtained differentially expressed genes and differentially methylated genes to obtain a molecular protein-protein interaction (PPI) network;
performing gene clustering with the molecular PPI network to identify target genes, comprising:
calculating the betweenness centrality, closeness centrality, average shortest path length and clustering coefficient of protein encoded by the differentially methylated genes and the differentially expressed genes in the molecular PPI network to obtain positions and relationships of the differentially methylated genes and the differentially expressed genes in molecular PPI network structure;
based on a K-means clustering method, carrying out feature selection according to the differences of the betweenness centrality, closeness centrality, average shortest path length and clustering coefficient, and dividing the differentially methylated genes and the differentially expressed genes into clusters with similar network features;
taking a cluster with the maximum sum of the betweenness centrality of nodes as a target cluster, and taking genes in the target cluster as the target genes.

2. The gene mining method based on transcriptome and DNA methylome according to claim 1, wherein tools for the gene function enrichment comprise DAVID, Enrichr, Metascape and GSEA;
reference databases for the gene function enrichment comprise GO, KEGG and Reactome.

3. The gene mining method based on transcriptome and DNA methylome according to claim 1, wherein the molecular PPI network is a molecular PPI network of genes contained in an enrichment pathway shared by the differentially expressed genes and the differentially methylated genes.

4. The gene mining method based on transcriptome and DNA methylome according to claim 1, wherein the betweenness centrality comprises the following formula:
$${C}_{B} ( υ ) = {\sum }_{δ \neq υ \neq t} \frac{d ( υ , u )}{{σ}_{ST}} ;$$
wherein C_{B}(υ) is the betweenness centrality of a node υ in the molecular PPI network, σ_{ST} is the number of shortest paths from a node δ to a node t in the molecular PPI network, d(υ,u) is the number of shortest paths from the node υ to other nodes in the molecular PPI network, and u is traversal from the node υ to other nodes in the molecular PPI network.

5. The gene mining method based on transcriptome and DNA methylome according to claim 1, wherein the closeness centrality comprises the following formula:
$${C}_{C} ( υ ) = \frac{1}{{\sum }_{u} d ( υ , u )} ;$$
wherein C_{C}(υ) is the closeness centrality of the node υ in the molecular PPI network, d(υ,u) is the number of shortest paths from the node υ to other nodes in the molecular PPI network, and u is traversal from the node υ to other nodes in the molecular PPI network.

6. The gene mining method based on transcriptome and DNA methylome according to claim 1, wherein the average shortest path length comprises the following formula:
$$L = \frac{1}{n ( n - 1 )} {\sum }_{i \neq j} {d}_{i j} ;$$
wherein Lis average shortest path length, n is the total number of nodes in the molecular PPI network, and dᵢⱼ is shortest path length from a node i to a node j in the molecular PPI network.

7. The gene mining method based on transcriptome and DNA methylome according to claim 1, wherein the clustering coefficient comprises the following formula:
$$C ( υ ) = \frac{2 \times number of triangles centered on υ}{degree of υ \times ( degree of υ - 1 )} ;$$
wherein C(υ) is the clustering coefficient of the node υ in the molecular PPI network, number of triangles centered on υ is the number of triangles centered on the node υ, and degree of υ is the degree of the node υ in the molecular PPI network.

8. A gene mining system based on transcriptome and DNA methylome, comprising:
a sample acquisition module used for acquiring samples;
a differentially expressed gene acquisition module used for performing transcriptome sequencing on the samples and analyzing data obtained by the sequencing to obtain differentially expressed genes;
a differentially methylated gene acquisition module used for performing DNA methylation sequencing on the samples and analyzing data obtained by the sequencing to obtain differentially methylated genes, comprising:
comparing DNA methylation data with reference genome to obtain basic methylated signals;
based on a Bayes machine learning method, calculating Hellinger divergence by using the DNA methylation level of the samples and the DNA methylation level of control samples with reference to the DNA methylation level of control samples, and fitting a statistical model by the Hellinger divergence according to the Akaike information criterion and the cross-validation correlation coefficient of nonlinear regression;
distinguishing methylated signals and background variation signals by the statistical model to screen out cytosine positions with methylated signals, and determining differentially methylated positions according to the cytosine positions with methylated signals and thresholds of the differentially methylated positions;
a molecular PPI network construction module used for performing gene function enrichment on the obtained differentially expressed genes and differentially methylated genes to obtain a molecular PPI network;
a target gene identification module used for performing gene clustering with the molecular PPI network to identify target genes, comprising:
calculating the betweenness centrality, closeness centrality, average shortest path length and clustering coefficient of protein encoded by the differentially methylated genes and the differentially expressed genes in the molecular PPI network to obtain positions and relationships of the differentially methylated genes and the differentially expressed genes in molecular PPI network structure;
based on a K-means clustering method, carrying out feature selection according to the differences of the betweenness centrality, closeness centrality, average shortest path length and clustering coefficient, and dividing the differentially methylated genes and the differentially expressed genes into clusters with similar network features;
taking a cluster with the maximum sum of the betweenness centrality of nodes as a target cluster, and taking genes in the target cluster as the target genes.
